(19) United States
(12) Patent Application Publication
(10) Pub. No.: US 2008/0300399 A1
(43) Pub. Date: Dec. 4, 2008

(54) PROCESSES RELATED TO MAKING CAPECITABINE

(76) Inventor: Gerrit J. B. Ettema, Nijmegen (NL)

Correspondence Address: SYNTHON IP INC, 7130 HERITAGE VILLAGE PLAZA, STE 202, GAINESVILLE, VA, 20155, US

(21) Appl. No.: 12/131,178

(22) Filed: Jun. 2, 2008

Related U.S. Application Data
(60) Provisional application No. 60/941,374, filed on Jun. 1, 2007.

(51) Int. Cl. C07H 19/06

(52) U.S. Cl. 536/28.51; 536/28.52

(57) ABSTRACT

An intermediate (2) useful in making capecitabine can be formed without the use, or presence, of a silylation agent.

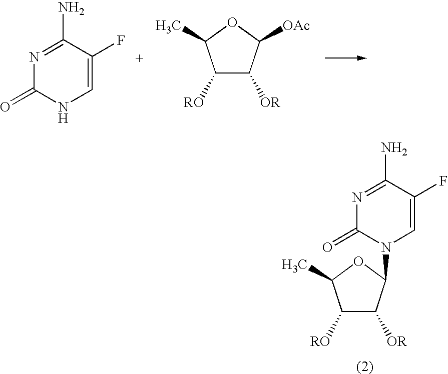

This application claims the benefit of priority under 35 U.S.C. § 119(e) from prior U.S. provisional application Ser. No. 60/941,374, filed Jun. 1, 2007, the entire contents of which are incorporated herein by reference.

Capecitabine, chemically 5′-Deoxy-5-fluoro-N4-(pentyloxycarbonyl)cytidine of the formula (1)

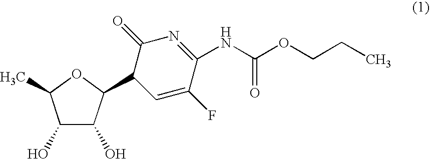

is an orally-administered pharmaceutically active compound developed for the treatment of various types of cancer. It is a prodrug, which passes intact through the intestinal mucosa of a patient and is activated by a cascade of three enzymes to give intra-tumoral release of the parent compound, 5-fluorouracil (5-FU), a known antitumor agent.

Capecitabine was generically disclosed in U.S. Pat. No. 4,966,891 and specifically disclosed in U.S. Pat. No. 5,472,949. In pharmaceutical compositions, it is marketed under the brand name XELODA® by Roche Laboratories Inc. (USA).

Various synthetic processes leading to capecitabine are known in the prior art. The key step in many of them comprises the introduction of a n-pentyloxycarbonyl side chain to the amino group. One of the processes starts from an intermediate compound of the formula (2) having, prior to the introduction of the pentyloxycarbonyl side chain on the nitrogen in position 4, the OH-groups of the furane ring protected by a hydroxy-protecting radical R (EP 602454, U.S. Pat. No. 5,472,949). The typical example of such intermediate is the diacetyldoxifluridine (5′-deoxy-2′,3′di-O-acetyl-5-fluorocytidine), a compound of the formula (2a)

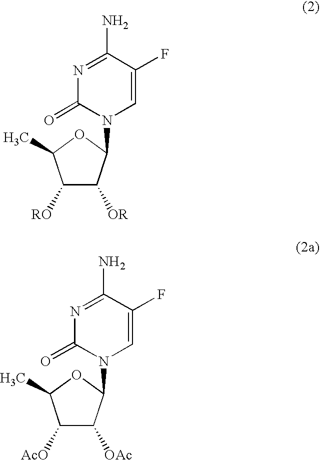

The compound of (2a), and more generally the compound of formula (2), can be converted into capecitabine in two steps. In the first step the compound of formula (2a) is reacted with n-pentylchloroformate in the presence of an organic base such as pyridine to form bis-acetylated capecitabine of formula (3a). In a second step, the compound (3a) is deprotected by and alkaline hydrolysis to yield capecitabine. The process is shown in the following scheme.

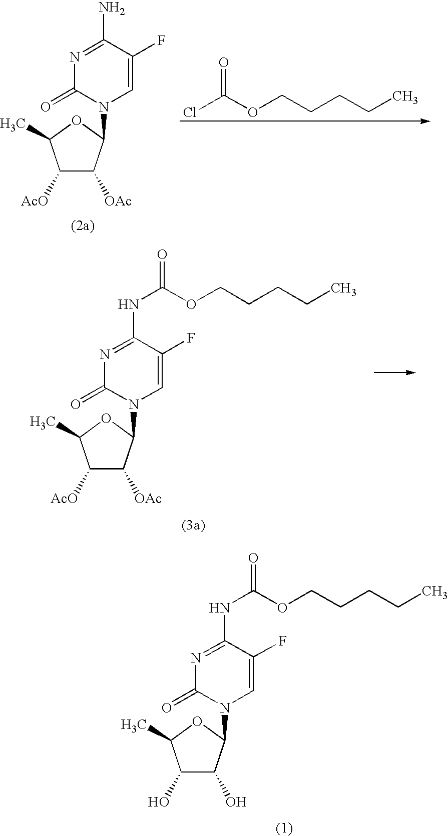

In a later variant of this process, (US 2005/137392) the acetylated fluorocytidine (2a) is first silylated (on the NH2-group or on the C═O group) and then reacted with the n-pentylchloroformate. Afterward the silyl-groups and hydroxy-protecting groups are removed. This variation is purported to enhance the selectivity of the overall process.

The conceptual process for making the starting intermediate of the formula (2) is a coupling of 5-fluorocytosine, the compound of formula (4), with an O-acetylated 5-deoxy-β-D-ribofuranose of the formula (5).

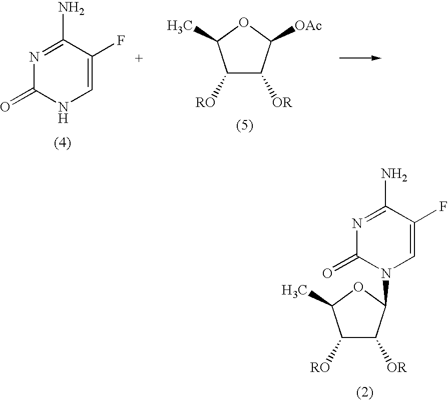

In practice, however, the 5-fluorocytosine is first treated with a silylation agent such as hexamethyldisilazane, trimethylsilyl chloride, etc.

For example, Shimma et al. (Bioorg. Med. Chem. 8 (2000), 1697-1706) shows treatment of compound (4) with HMDS (i.e., hexamethyldisilazane) in toluene before reacting it with 1,2,3-tri-O-acetyl-5-deoxy-β-D-ribofuranose (5a) (See Scheme 2 of Shimma et al.).

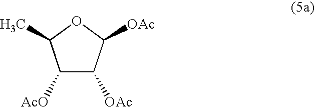

This treatment with hexamethyldisilazane was subsequently reported by Zheng et al. (Nuclear Medicine and Biology 31 (2004), 1033-1041), to form a 5-fluorocytosine trimethylsilyl derivative of (4). Thus, a silyl derivative of (4) was understood to be formed by the pretreatment with HMDS and this silyl derivative was subsequently reacted with the compound (5a) in Shimma et al. The reported yield in Shimma et al. of the product (2a) is 76%. A variation on the Shimma et al. pretreatment is reported in US 2005-137392, wherein the reaction with hexamethyldisilazane may be catalyzed by a triflic acid.

A second approach mentioned in EP 602 454 and U.S. Pat. No. 5,472,949, directly couples the trimethylsilylated derivative of the compound (4) with the compound (5a) in the presence of an in situ generated trimethyl silyl iodide as the required Lewis acid as described by Matsuda et al. (Synthesis 1981, p. 748). The reported yield is 49% after purification and recrystallization.

According to the above methods, the compound of formula (4) is treated with and/or modified by a silylation agent before it is converted to a compound of formula (2). While such processes are suitable, it would be desirable to have an alternative and/or simpler process for making the compound of formula (2).

### SUMMARY OF THE INVENTION

The present invention relates to a process for making the compound of formula (2) and optionally further converting it into capecitabine. Accordingly a first aspect of the invention relates to a process which comprises reacting, in the presence of a Lewis acid and in the absence of a silylation agent, a compound of formula (4) with a compound of formula (5) to form a compound of formula (2)

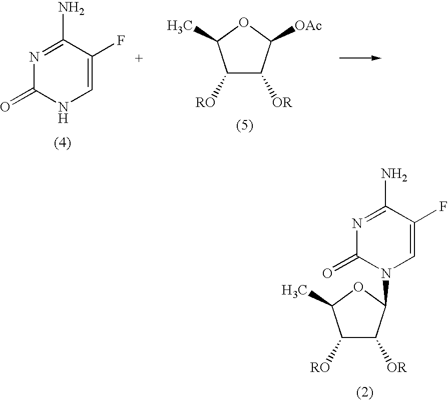

wherein each R represents hydrogen, an OH-protective group such as an acetyl, trifluoroacetyl, benzoyl, benzyl, or trityl group, or both R moieties join together to form a ring, such as an isopropylidene group. Generally the compound of formula (5) is the compound of formula (5a) resulting in the formation of a compound of formula (2a).

Another aspect of the invention relates to converting the above formed compound of formula (2) into capecitabine of formula (1).

### DETAILED DESCRIPTION OF THE INVENTION

The present invention is based on the finding that the glycosidation of the compound of formula (4) by the O-acetylated compound of the formula (5) in the presence of a Lewis acid catalyst, can proceed without the presence or use of a silylation agent. Contrary to the above-mentioned documents, which routinely teach the pretreatment of (4) with a silylation agent, e.g. treating with hexamethyldisilazane, or the outright use of a silylated derivative of the compound (4), e.g., trimethylsilylated derivative, it has been discovered that compound (4) can be directly reacted with a compound of formula (5), and particularly with the compound of formula (5a), without such silylation agent or pretreatment to obtain compound (2) in good yield and purity.

For purposes of the present invention, carrying out the reaction between compounds (4) and (5) “in the absence of a silylation agent” means that neither compounds (4) or (5) are silylated to carry out the coupling reaction, nor is a silylation agent present in the reaction medium during this coupling reaction. Thus in the process of the invention the compound of formula (4) is not subjected to treatment with a silylation agent, particularly with hexamethyldisilazane, prior to (or concurrently with) it being contacted with the compound of formula (5). Likewise, it is a compound of formula (4) that is reacted and not a silylated derivative thereof.

Avoiding the silylation step has advantages from an economical aspect (simplifying the process without using an expensive silylation agent) and an ecological aspect (no need of a disposal of a silicon-containing wastes). Also, the fluorocytosine of the formula (4) has more than one reactive site for the silylation. Accordingly, the product of silylation may comprise a mixture of different regioselectively silylated compounds of a different reactivity, which would decrease the batch-to-batch reliability of the overall process in terms of yield and quality of the product. Alternatively, it is possible that in some processes the expected silylation reaction does not occur at all. In either event, the present invention seeks a simpler, yet reliable process that is economically attractive by avoiding the silylation of the compound of formula (4) and the silylation agent used therefor.

In general, the process of the present invention comprises reacting, in the presence of a Lewis acid and in the absence of a silylation agent, a compound of formula (4) with a compound of formula (5) to form a compound of formula (2).

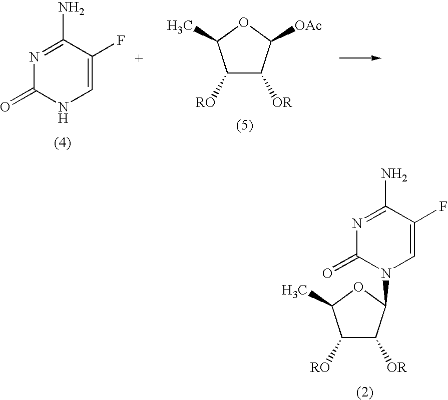

Each moiety R in formula (5) independently represents hydrogen, an OH-protecting group, or both R moieties join together to form a ring. The OH-protecting groups include acetyl, trifluoroacetyl, benzoyl, benzyl, and trityl group. When both R moieties join together to form a ring, the R moieties together represent 1 to 3 carbon atoms. The resulting rings include an isopropylidene ring. The same definition of R applies to the compound of formula (2). Typically R represents an acetyl group, which corresponds to the formula (5a). The compounds of formula (5) can be made by methods known in the art and/or by analogous procedures thereto, by workers of ordinary skill. In particular the compound (5a) is a known compound that may be obtained by various processes known in the art (see, e.g., Sairam et al, Carbohydrate Research 338 (2003), 303-306, Zheng et al, Nuclear Medicine and Biology 31 (2004), 1033-1041). The compound (4) is a commercially available compound.

The reaction is carried out in the presence of a Lewis acid which facilitates the coupling reaction, generally a condensation reaction, between (4) and (5). Suitable Lewis acids include stannic chloride, ferric chloride, cesium chloride, dimethyl tin(IV) chloride, titanium tetrachloride and triflic acid. Generally stannic chloride is used.

The amount of the Lewis acid is typically 1 to 1.5 molar equivalents with respect to the amount of the compound of formula (4). Likewise the compound of formula (5) is generally used in equimolar or molar excessive amounts relative to the amount of the compound of formula (4). Typically the molar ratio of the reagents of formula (4) and (5), respectively, is from 1:1 to 1:1.2.

The reaction can be carried out in a solvent; i.e., a liquid reaction medium, generally an organic solvent. Preferably the solvent is a non-protic organic solvent, including dichloromethane, acetonitrile, toluene, dimethylsulfoxide and mixtures thereof, but is not limited thereto. Advantageously, water immiscible solvent systems are preferred due to the subsequent workup.

The suitable reaction temperature is generally in the range from 0° to 40° C. and conveniently is room or ambient temperature. The reaction course may be monitored by a suitable analytical technique, for instance HPLC.

The reaction product, the compound of formula (2), may be isolated from the reaction mixture; however it can also be used in the subsequent reactions without the isolation as will be shown below. As used herein “isolation” is used in a narrow sense, meaning to obtain the desired compound in a substantially solid state, such as a residue or a precipitate that is substantially free of solvent and other reagents; e.g., at least 75% pure. A suitable isolation process comprises treating the reaction mixture with water, extraction of the product into an organic phase and separating the product from the organic phase such as by removing the solvent and/or precipitating and filtering off the solid product. The isolated product may be subsequently purified, e.g., by chromatography or by a recrystallization from a suitable solvent.

The condensation or coupling between (4) and (5) introduces a new chiral center into the molecule. Fortunately the process proceeds with high stereospecificity, i.e., the formed C—N bond between the sugar moiety and the pyrimidine base is in the desired configuration. As a result, a product with low amounts of the unwanted enantiomer is formed and the overall purity of the product after a single recrystallization may be higher than 95%, advantageously higher than 99%.

The compound of formula (2), and particularly the compound of formula (2a), is a useful chemical that may serve as a starting material for the synthesis of capecitabine of formula (1). It may be converted into capecitabine by known processes as disclosed in U.S. Pat. No. 5,472,949.

Generally the conversion involves reacting the compound (2) with n-pentylchloroformate in an inert solvent (e.g. dichloromethane) in the presence of an organic base, which is advantageously pyridine or 3-picoline to form “protected capecitabine”; i.e., a compound of formula (3) and more advantageously, the compound (3a).

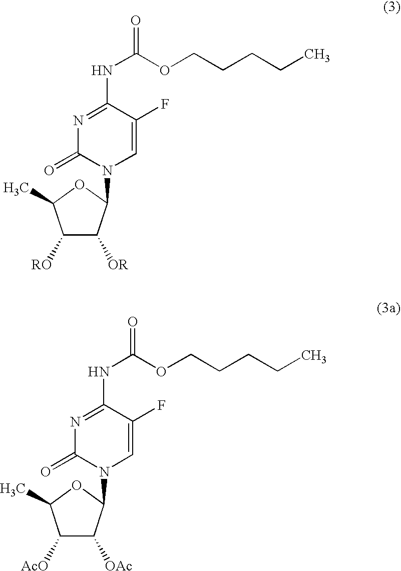

The product of formula (3) may be isolated from the reaction mixture by processes disclosed in the prior art and purified, if necessary.

The protected capecitabine, the final intermediate (3), is converted into capecitabine by removing the protective moiety R by a suitable deprotection method, which is advantageously an alkaline hydrolysis. After isolating from the reaction mixture, the capecitabine can be crystallized from a suitable solvent, e.g. from ethyl acetate/hexane as described in literature, to provide a crystalline capecitabine. Alternatively, the isolated capecitabine may be dissolved in water and the solution freeze-dried to provide an amorphous capecitabine.

As the inventive process can provide the compound of formula (2) in a high conversion and high purity, the whole process of making capecitabine from the compound (4) may proceed in a “one-pot” arrangement (i.e., without the isolation of intermediates (2) or (3)) with good yields and with sufficient purity of the final product.

In an example of such one-pot process, the reaction mixture comprising the product of formula (2) provided by the inventive condensation of compounds (4) and (5), i.e. without the presence of, or a pre-treatment with, a silylation agent, is typically concentrated to lower volumes. Then n-pentylchloroformate and a base (e.g. pyridine) are added, allowed to react, and finally a solution of a hydroxide (e.g. NaOH) in a suitable solvent is added. After the hydrolysis is complete, the mixture is neutralized, and the capecitabine product is extracted by a water immiscible organic solvent. After removal of the solvent, the crude capecitabine may be recrystallized, e.g. from an ethyl acetate-hexane mixture.

This one-pot process can result in yields of 50-60% or more with purity higher than 95%. Such yields are comparative to those disclosed in US 2005-137392 for a similar process, but superior in purity and simpler in arrangement.

The invention is further illustrated by the following non-limiting examples.

### EXAMPLES

### Example 1

### Compound (2a)

2.62 g of 5-fluorocytosine was added to 33 ml of CH₂Cl₂ and 5.93 g 5-deoxy-1,2,3-tri-O-acetyl-β-d-ribofuranose was added. The suspension was stirred and cooled to 0-4° C. in an ice-bath. 6.25 g of stannic chloride was added dropwise in about 10 minutes. The mixture was allowed to heat up to room temperature and stirred for 1 hour. An almost clear solution was obtained. 10.1 g of sodium bicarbonate was added and 3.5 ml of water was added dropwise (gas formation). The mixture was stirred at room temperature overnight. The insoluble material was filtered off. The filtrate was washed with saturated aqueous sodium bicarbonate solution. The organic layer was dried on Na₂SO₄ and filtered. The solvent was removed under reduced pressure. The residue was co-evaporated with 5 ml of 2-Propanol and a solid was obtained.

Yield: 6.4 g

The solid was recrystallized from 16 ml of 2-propanol, stirred after cooling to room temperature for 16 hours.

Melting Point: 190.1-191.2° C.

NMR confirmed the structure.

HPLC purity: higher than 99.5%.

### Example 2

### Compound 3a

0.8 g of 2′,3′-di-O-acetyl-5′-deoxy-5-fluoro-cytidine (Example 1) was dissolved in 2 ml dry Cl₂ and 0.4 ml of pyridine was added. The mixture was cooled on an ice-water bath. 0.5 ml of n-pentylchloroformate was added dropwise in 5 minutes. The clear colorless solution was allowed to warm up to room temperature upon stirring. A suspension was formed. After 30 minutes, the reaction mixture was concentrated at reduced pressure. 5 ml of diethylether was added to the residue. The resulting suspension was filtered. The filtrate was concentrated under reduced pressure. Yield: 1.3 g (colorless oil).

### Example 3

### Capecitabine Compound (1)

1.2 g of the oil from the Example 2 was dissolved in 3 ml of methanol. A solution of 0.4 g NaOH in 2 ml water was added at 0° C. After stirring for 30 minutes at 0° C., the pH was adjusted to about 5 by addition of concentrated HCl. Then, 10 ml dichloromethane and 5 ml water were added. Mixed for 5 minutes. The layers were separated. The organic layer was washed with 5 ml of water, dried on Na₂SO₄ and concentrated under reduced pressure. To the oil was added 1 ml of ethyl acetate. To the resulting solution 2 ml of n-heptane was added. An oily precipitate was formed. Seeded with a seed of capecitabine crystals, the oil slowly solidified. After 2 hours the solid was filtered off and dried in a vacuum oven at 40° C. for 16 hours.

Yield: 0.58 g

NMR: confirmed the structure

HPLC: purity >99.8%

### Example 4

### Capecitabine by “One Pot” Process

2.58 g of 5-fluorocytosine and 5.93 g of 5-deoxy-1,2,3,-tri-O-acetyl-β-D-furanoside were added to 33 ml of dichloromethane, the mixture was cooled to 0-4° C., stirred and 6.25 g of stannic chloride was added dropwise in about 10 minutes. The mixture was allowed to heat up to room temperature and stirred for about 2.5 hours. 10.1 g of sodium bicarbonate was added. 35 ml of water was added dropwise over a period of 20 minutes. (CO₂ formation) The reaction mixture was stirred overnight. About 2 g of Na₂SO₄ was added to the organic phase and stirred for 30 minutes. The solid was filtered off, washed with 10 ml of dichloromethane. The combined filtrates were reduced in volume to about 20 ml. by reduced pressure. To the resulting solution was added 3.9 ml of pyridine. The mixture was cooled to 0-4° C. (ice-bath). 4.9 ml n-pentylchloroformate was added dropwise. The ice-bath was removed and after 40 minutes 13 ml of methanol was added. The mixture was cooled on an ice bath. A solution of 4.68 g of NaOH in 6.5 ml of water was added dropwise in 10 minutes. Then 9.8 ml of concentrated HCl was added dropwise. After addition the pH was about 5 (pH-paper). 65 ml of dichloromethane and 13 ml water was added. The layers were mixed and allowed to separate. The organic layer was washed with 13 ml of water, dried on Na₂SO₄ and filtered. The filtrate was evaporated to dryness under reduced pressure. The oily residue was dissolved in 8.6 ml of ethyl acetate and 17 ml n-hexane was added. A solid was formed. After stirring overnight, the solid was isolated by filtration and washed with a mixture of 8.6 ml ethyl acetate and 17 ml n-hexane. Dried in a vacuum oven at 40° C.

Yield: 4.3 g (60%), purity HPLC: 98.7%

Each of the patents, patent applications, and journal articles mentioned above are incorporated herein by reference in their entirety. The invention having been thus described, it will be obvious to the worker skilled in the art that the same may be varied in many ways without departing from the spirit of the invention and all such modifications are included within the scope of the present invention as set forth in the following claims.

I claim:

1. A process, which comprises reacting, in the presence of a Lewis acid and in the absence of a silylation agent, a compound of formula (4) with a compound of formula (5) to form a compound of formula (2): 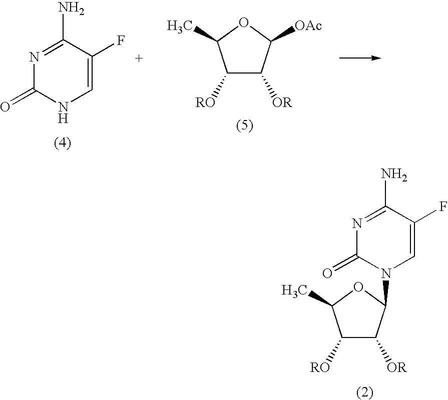
wherein each R represents hydrogen, an OH-protecting group, or both R moieties join together to form a ring.

2. The process according to claim 1, wherein R represents an OH-protecting group selected from the group consisting of an acetyl, trifluoroacetyl, benzoyl, benzyl, and trityl group.

3. The process according to claim 1, which further comprises converting said compound of formula (2) into capecitabine.

4. The process according to claim 3, wherein said converting comprises:
(i) reacting said compound of formula (2) with n-pentylchloroformate in the presence of an organic base to form protected capecitabine; and
(ii) deprotecting said protected capecitabine to form capecitabine.

5. The process according to claim 4, wherein said reacting and converting steps are carried out in a one pot process.

6. The process according to claim 1, wherein said compound of formula (5) is a compound of formula (5a) and the compound formed of formula (2) is a compound of formula (2a):

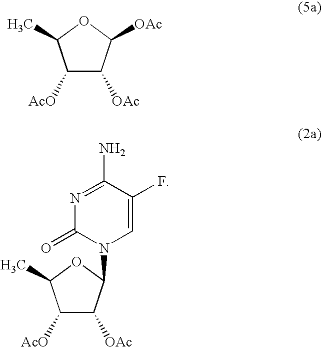

7. The process according to claim 6, which further comprises converting said compound of formula (2) into capecitabine.

8. The process according to claim 6, which further comprises:
(i) reacting said compound of formula (2a) with converting with n-pentylchloroformate in the presence of an organic base to form a compound of formula (3a); and
(ii) deprotecting said compound of formula (3a) to form capecitabine.

9. The process according to claim 8, wherein said compounds of formula (2a) and (3a) are not isolated.
